(19) United States
(12) Patent Application Publication
(10) Pub. No.: US 2011/0028805 A1
(43) Pub. Date: Feb. 3, 2011

(54) INFORMATION PROCESSING APPARATUS, METHOD, AND PROGRAM

(75) Inventor: Takahiro YAMAZAKI, Saitama (JP)

Correspondence Address: OBLON, SPIVAK, MCCLELLAND MAIER & NEUSTADT, L.L.P., 1940 DUKE STREET, ALEXANDRIA, VA, 22314, US

(73) Assignee: Sony Corporation, Tokyo (JP)

(21) Appl. No.: 12/837,927

(22) Filed: Jul. 16, 2010

(30) Foreign Application Priority Data
Jul. 28, 2009 (JP) 2009-175442

(51) Int. Cl. A61B 5/00; H04N 13/00; A61B 5/024; A61B 5/11; A61B 5/026

(52) U.S. Cl. 600/307; 348/42; 600/508; 600/300; 348/E13.001

(57) ABSTRACT

An information processing apparatus includes: reproduction means for reproducing 3D content; and detection means for detecting a biological signal associated with a viewer who is viewing the 3D content, wherein the reproduction means attenuates the 3D effect of the 3D content when the viewer's biological signal produced when the 3D content is being reproduced exceeds a threshold value.

### BACKGROUND OF THE INVENTION

1. Field of the Invention

The present invention relates to an information processing apparatus, an information processing method, and an information processing program, and particularly to an information processing apparatus, an information processing method, and an information processing program that can prevent 3D sickness.

2. Description of the Related Art

In recent years, a system that allows a viewer to observe 3D (3 Dimensions) stereoscopic video images has been becoming popular.

The viewer who is viewing 3D stereoscopic video images, however, complains of sickness (hereinafter referred to as 3D sickness) in some cases. In this case, the viewer either continues to view the 3D stereoscopic video images even though he/she is suffering from the 3D sickness or stops viewing based on his/her own decision.

JP-A-2000-339490 discloses a technology for checking whether a viewer is suffering from “sickness” by acquiring a biological signal associated with the viewer and processing the biological signal in a virtual reality system (hereinafter referred to as a VR system).

WO 04/029693 discloses a wearable display technology for reducing the degree of “sickness” by temporarily storing image information in an internal storage device, processing the image information in accordance with the motion in images, and presenting the processed image information to a viewer.

### SUMMARY OF THE INVENTION

The technology described in JP-A-2000-339490 is, however, a technology applied to a VR system, as described above, and used to reduce the speed at which the chair on which the viewer is seated is moved or rotated and lower the brightness and the contrast of images when the viewer is suffering from “sickness.” The technology described in JP-A-2000-339490 may therefore not be directly applied to a 3D system that does not involve any movement or rotation.

The technology described in WO 04/029693 is a technology for providing in advance image information for reducing the degree of sickness. The technology may therefore not be applied to a case where a viewer who is viewing ordinary images is suffering from 3D sickness.

Thus, it is desirable to prevent 3D sickness.

An information processing apparatus according to an embodiment of the invention includes reproduction means for reproducing 3D content and detection means for detecting a biological signal associated with a viewer who is viewing the 3D content, and the reproduction means attenuates the 3D effect of the 3D content when the viewer's biological signal produced when the 3D content is being reproduced exceeds a threshold value.

The information processing apparatus may further include storage means for storing a standard value of the viewer's biological signal and viewer information representing the characteristics of the viewer, the standard value and the viewer information related to each other, and the reproduction means may change the threshold value based on a viewing pattern related to a pattern according to which at least one of video and audio signals representing the 3D content changes, the viewing pattern stored in advance on a 3D content storage medium on which the 3D content is stored.

The viewer information may contain at least the age of the viewer, and when a plurality of viewers are viewing the 3D content and the viewer whose biological signal shows a value greater than the threshold value is of a predetermined age or under, the reproducing means places a priority on the viewer and attenuates the 3D effect accordingly.

The reproduction means may further change the threshold value over the period during which the 3D content is reproduced.

The reproduction means may transmit information obtained by relating to one another the viewer information, the viewer's biological signal, and the time along which the 3D content is reproduced via a network.

An information processing method and an information processing program of the information processing apparatus according to another embodiment of the invention are a method and a program corresponding to the information processing apparatus according to the embodiment of the invention described above.

The another embodiment of the invention involves reproducing 3D content, detecting a biological signal associated with a viewer who is viewing the 3D content, and attenuating the 3D effect of the 3D content when the viewer's biological signal produced when the 3D content is being reproduced exceeds a threshold value.

According to another embodiment of the invention, 3D sickness can be prevented.

### BRIEF DESCRIPTION OF THE DRAWINGS

FIG. 1 is a block diagram showing an exemplary configuration of a 3D stereoscopic video image apparatus as an information processing apparatus according to an embodiment to which the invention is applied;

FIG. 2 describes 3D sickness associated with viewers versus the time along which 3D content is reproduced;

FIG. 3 shows an example of viewer information;

FIG. 4 describes the relationship between a viewing pattern and a sickness detection threshold value;

FIG. 5 shows an example of how to seta sickness detection threshold value;

FIG. 6 is a flowchart describing operation of an exemplary information processing method to which an embodiment of the invention is applied; and

FIG. 7 is a block diagram showing an exemplary configuration of a computer that controls an information processing apparatus to which an embodiment of the invention is applied.

### DESCRIPTION OF THE PREFERRED EMBODIMENTS

### Exemplary Configuration of 3D Stereoscopic Video Image Apparatus

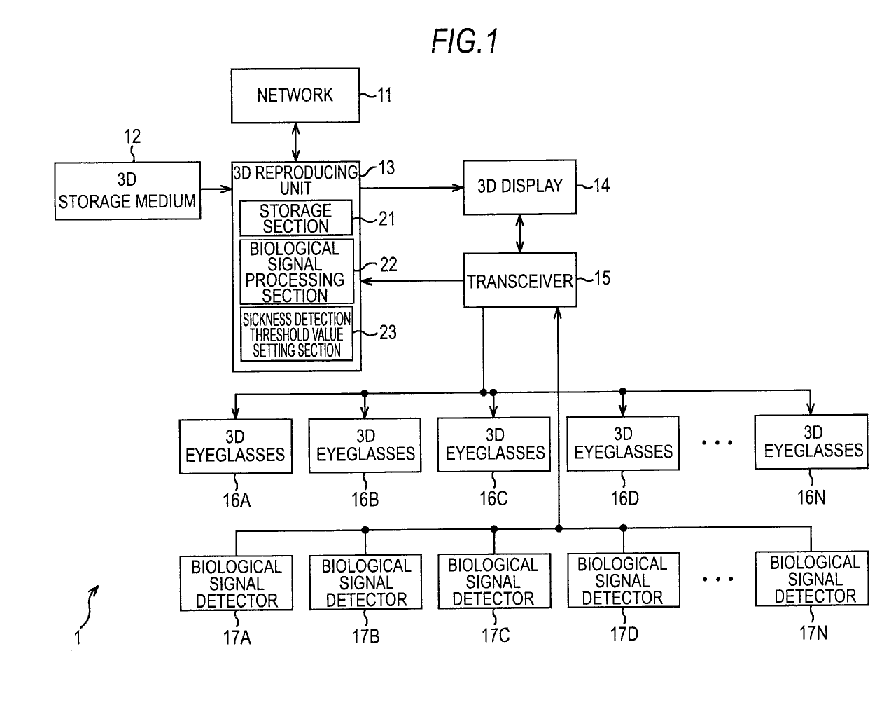

FIG. 1 is a block diagram showing an exemplary configuration of a 3D stereoscopic video image apparatus as an information processing apparatus according to an embodiment of the invention.

Methods for conveying 3D stereoscopic video images include a method for conveying images having parallax to the eyes of a viewer who wears a pair of polarized eyeglasses, a pair of eyeglasses with liquid crystal shutters, or any other special eyeglasses and a method using an autostereoscopic display. An autostereoscopic display is a display providing parallax to the eyes of a viewer, and the method using the autostereoscopic display, for example, uses a parallax barrier that allows separate light rays to be incident on the right and left eyes.

Any method for conveying 3D stereoscopic video images can be employed in the present embodiment. The following description will be made with reference to a case where a method using a pair of eyeglasses with liquid crystal shutters is employed.

A 3D stereoscopic video image apparatus 1 in the example shown in FIG. 1 includes a 3D storage medium 12, a 3D reproducing unit 13, a 3D display 14, a transceiver 15, 3D eyeglasses 16A to 16N, and biological signal detectors 17A to 17N. The 3D stereoscopic video image apparatus 1 is connected to a network 11 represented by the Internet as necessary.

The 3D storage medium 12 is formed, for example, of a BD (Blu-Ray® Disc) and stores content containing a 3D stereoscopic video image signal and a corresponding audio signal (hereinafter referred to as 3D content).

The 3D reproducing unit 13 reads and reproduces the 3D content stored on the 3D storage medium 12 and displays the 3D content on the 3D display 14. The 3D reproducing unit 13 includes a storage section 21, a biological signal processing section 22, and a sickness detection threshold value setting section 23. The storage section 21, the biological signal processing section 22, and the sickness detection threshold value setting section 23 will be described later.

The 3D display 14 displays the 3D content reproduced by the 3D reproducing unit 13. The 3D display 14 transmits via the transceiver 15 a control signal synchronized with the video signal to the 3D eyeglasses 16A to 16N (N is an integer greater than or equal to one) in accordance with the 3D content. That is, the 3D display 14 transmits to the 3D eyeglasses 16A to 16N a control signal that drives the liquid crystal shutters in such a way that video images delivered to the right and left eyes of the viewers are alternately blocked in accordance with the video signal. The viewers can thus visually recognize 3D stereoscopic video images when right and left video images having parallax are incident on their right and left eyes.

The transceiver 15 transmits the control signal received from the 3D display 14 to the 3D eyeglasses 16A to 16N. The transceiver 15 has an infrared light generator or a radio wave transmitter (not shown) incorporated therein and transmits the control signal in the form of infrared light or radio wave.

The transceiver 15 also has a detector (not shown) incorporated therein that detects infrared light or a radio wave. The transceiver 15 uses the detector to receive viewers' biological signals outputted from the biological signal detectors 17A to 17N (N is an integer greater than or equal to one). The transceiver 15 then outputs the received viewers' biological signals to the 3D reproducing unit 13.

To transmit and receive the biological signals and the control signal, for example, the system disclosed in JP-A-2007-235880 can be used. The system carries out data communication between information transmitting terminals by irradiating light beams containing specific information into the atmosphere.

The 3D eyeglasses 16A to 16N receive the control signal synchronized with the video signal from the transceiver 15. The 3D eyeglasses 16A to 16N are worn by the viewers respectively who are viewing the 3D content. The number of the 3D eyeglasses 16A to 16N to be prepared therefore corresponds to the number of viewers who are simultaneously viewing the 3D content. The 3D eyeglasses 16A to 16N are hereinafter collectively referred to as the 3D eyeglasses 16 when they are not necessary to be distinguished from one another.

The biological signal detectors 17A to 17N detect biological signals associated with each of the viewers who are viewing the 3D content. Each of the biological signal detectors 17A to 17N has an infrared light generator or a radio wave transmitter (not shown) incorporated therein, as in the case of the transceiver 15. The biological signal detectors 17A to 17N transmit the detected biological signals to the transceiver 15 in the form of infrared light or radio wave. The biological signal detectors 17A to 17N are worn by each of the viewers who are viewing the 3D content. The number of the biological signal detectors 17A to 17N to be prepared therefore corresponds to the number of viewers who are simultaneously viewing the 3D content, as in the case of the 3D eyeglasses 16. The biological signal detectors 17A to 17N are hereinafter collectively referred to as the biological signal detectors 17 when they are not necessary to be distinguished from one another.

The 3D eyeglasses 16 and the biological signal detectors 17 are related to the respective viewers. That is, for example, a viewer A wears the 3D eyeglasses 16A and the biological signal detector 17A. A viewer B wears the 3D eyeglasses 16B and the biological signal detector 17B. In this way, a viewer wears a set of a pair of 3D eyeglasses 16 and a biological signal detector 17.

Each of the biological signal detectors 17 detects a biological signal described in JP-A-2000-339490.

That is, a first example of the signal detected by each of the biological signal detectors 17 is the number of breathing, the magnitude of breathing, irregularity of the breathing of the corresponding viewer obtained by measuring breathing of the viewer.

A second example of the signal detected by each of the biological signal detectors 17 is the magnitude of shaking of the center of gravity of the corresponding viewer obtained by measuring the center of gravity of the viewer.

A third example of the signal detected by each of the biological signal detectors 17 is an average instantaneous heart rate, the components contained in a breath during heart rate variation, and Mayer wave component during heart rate variation of the corresponding viewer obtained by measuring the heart beats of the viewer.

A fourth example of the signal detected by each of the biological signal detectors 17 is the magnitude of a specific frequency component of base line variation of the corresponding viewer obtained by measuring a cardiogram of the viewer.

A pair of 3D eyeglasses 16 and a biological signal detector 17 may separately be worn by a viewer or may be integrated into a pair of 3D eyeglasses 16 with a built-in biological signal detector 17. When a pair of 3D eyeglasses 16 with a built-in biological signal detector 17 is used, examples of the biological signals that are readily acquired include any change in blood flow rate and the state of perspiration of the viewer as well as any movement of the head (shaking of the center of gravity of the head) of the viewer measured with an acceleration sensor.

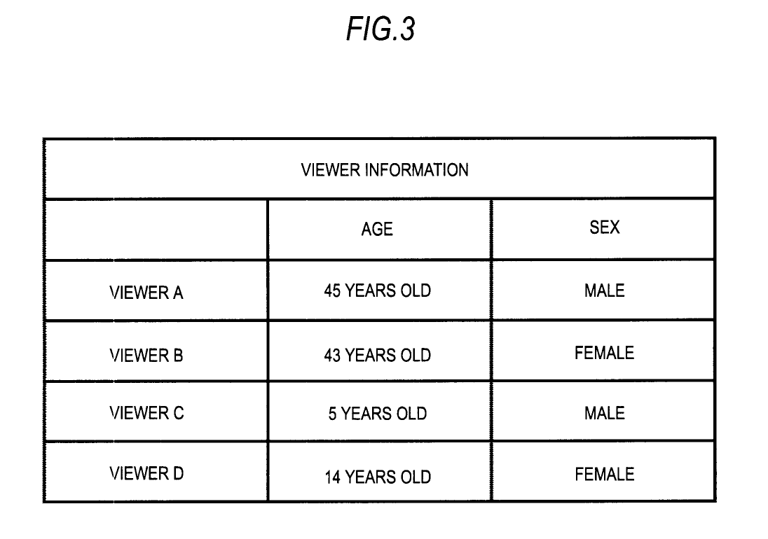

The storage section 21 stores information representing the characteristics of each viewer who wears a pair of 3D eyeglasses 16 and a biological signal detector 17. The information on each viewer stored in the storage section 21 is hereinafter referred to as viewer information. Specific examples of the viewer information will be described later with reference to FIG. 3.

The viewer information is inputted in advance, for example, by using a remote controller or any other suitable input means (not shown) associated with the 3D reproducing unit 13.

The storage section 21 further stores a viewer's standard biological signal received from the corresponding biological signal detector 17 via the transceiver 15. The standard biological signal used herein is a biological signal produced when the viewer is not viewing any 3D content. That is, before any 3D content is reproduced, the storage section 21 stores in advance viewer information associated with a viewer who wears a pair of 3D eyeglasses 16 and a biological signal detector 17 and a standard biological signal related to the viewer information.

The biological signal processing section 22 receives biological signals associated with the viewers from the biological signal detectors 17 via the transceiver 15. The biological signal processing section 22 analyzes the received viewers' biological signals to judge whether or not any of the viewers is suffering from 3D sickness. Specifically, for example, when head movement is used as the biological signal, the biological signal processing section 22 monitors information on head movement at all times provided from the biological signal detectors 17. When the magnitude of the head movement of a viewer per unit time exceeds a predetermined threshold value, the biological signal processing section 22 judges that the viewer is suffering from 3D sickness.

The sickness detection threshold value setting section 23 sets a threshold value to be compared with a biological signal in order to detect that the corresponding viewer is suffering from 3D sickness. That is, a biological signal associated with a viewer produced when the viewer is not viewing any 3D content is used as a standard value, and a value larger than the standard value by a predetermined value is used as the threshold value. The threshold value is hereinafter referred to as a sickness detection threshold value. As described above, the biological signal processing section 22 judges whether or not a viewer is suffering from 3D sickness based on the sickness detection threshold value set by the sickness detection threshold value setting section 23.

When a viewer is judged to be suffering from 3D sickness, the 3D reproducing unit 13 attenuates the 3D effect (the amount of stereoscopic effect of 3D video images) of the 3D content displayed on the 3D display 14. Alternatively, when a viewer is judged to be suffering from 3D sickness, the 3D reproducing unit 13 switches the video signal representing 3D content to a 2D (2 Dimensions) video signal or temporarily halts displaying the 3D video images. The attenuation of the 3D effect, the switching of a 3D content video signal to a 2D video signal, the temporary halt of 3D video image display, and other actions are hereinafter referred to as 3D sickness relieving actions.

The 3D reproducing unit 13 is connected to the network 11. The 3D reproducing unit 13 can therefore send a 3D content producer the viewer information, biological signals produced when the viewers are viewing 3D content and related to the viewer information, and the state of 3D sickness and other information of the viewers, for example, via the network 11.

The exemplary configuration of the 3D stereoscopic video image apparatus 1 as an information processing apparatus according to an embodiment of the invention has been described with reference to FIG. 1.

### [Example of 3D Sickness Detection]

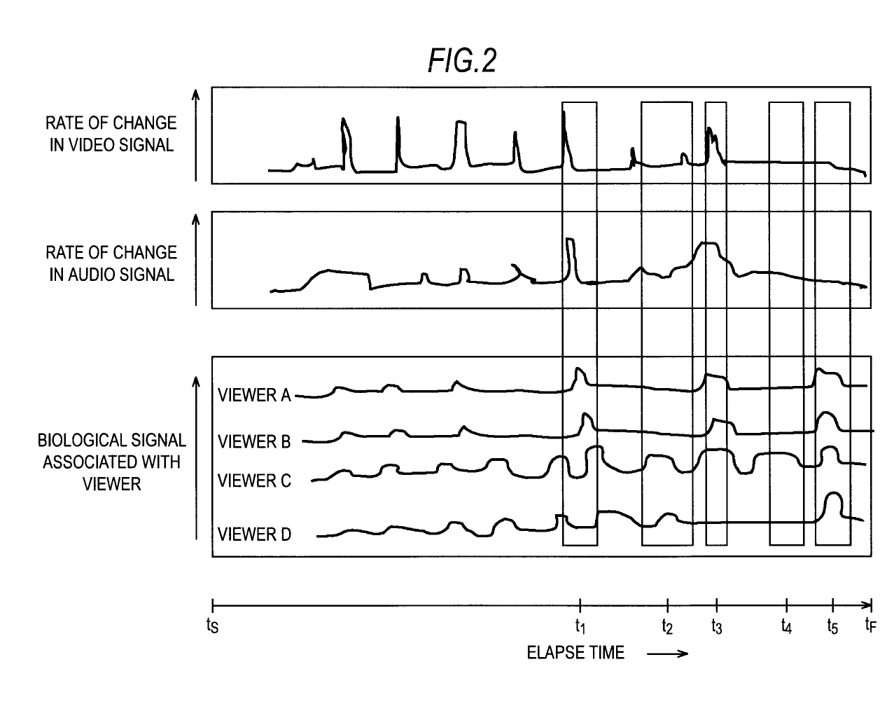

FIG. 2 describes change in viewers' biological signals verses change in video and audio signals representing 3D content.

The horizontal axis of FIG. 2 represents the time along which 3D content is reproduced. That is, when certain 3D content is reproduced, the reproduction of the 3D content starts from time tS and ends at time tF.

The graph in the upper portion of FIG. 2 shows the difference per unit time (rate of change) in brightness level of the video signal representing the 3D content versus the time along which the 3D content is reproduced. The vertical axis of the graph in the upper portion of FIG. 2 may be the rate of change in luminance of the video signal.

The graph in the middle portion of FIG. 2 represents the rate of change in the audio signal representing the 3D content versus the time along which the 3D content is reproduced.

The graphs in the lower portion of FIG. 2 represent the change in the viewers' biological signals versus the time along which the 3D content is reproduced. In the graphs in the lower portion of FIG. 2, a larger vertical change in the biological signal associated with any of the viewers means that the viewer is suffering from greater sickness.

The graphs in the lower portion of FIG. 2 show the biological signals associated with the viewers who are viewing the same 3D content.

Viewer information on viewers A to D corresponding to the graphs shown in the lower portion of FIG. 2 will be described with reference to FIG. 3.

As described above, the viewer information shown in FIG. 3 is stored in the storage section 21.

In FIG. 3, the viewer A wears the 3D eyeglasses 16A and the biological signal detector 17A. The viewer A is a 45-year-old male.

The viewer B wears the 3D eyeglasses 16B and the biological signal detector 17B. The viewer B is a 43-year-old female.

The viewer C wears the 3D eyeglasses 16C and the biological signal detector 17C. The viewer C is a 5-year-old male.

The viewer D wears the 3D eyeglasses 16D and the biological signal detector 17D. The viewer D is a 14-year-old female.

In the vicinities of time t1 and t3 in FIG. 2, the viewers A to D show large biological signal values. In the vicinities of the time t1 and t3, the rates of change in the video and audio signals are also high. That is, the 3D content presumably shows an exciting scene, such as an explosion scene, in the vicinities of the time t1 and t3.

In such a scene, the 3D effect of the 3D content tends to increase, and the viewers tend to be excited. The sickness detection threshold value setting section 23 therefore sets a larger sickness detection threshold value in such a scene. That is, the biological signal processing section 22 judges that the viewers are not suffering from 3D sickness even when they show larger biological signal values than those in other scenes. The 3D reproducing unit 13 therefore tends to take no 3D sickness relieving action.

In the vicinities of time t2 and t4, the rates of change in the video and audio signals are small. That is, the 3D content presumably does not particularly show an exciting scene in the vicinities of the time t2 and t4. The biological signals associated with the viewers A and B are small, whereas the biological signals associated with the viewers C and D are large. As described above, the viewer A is a 45-year-old male; the viewer B is a 43-year-old female; the viewer C is a 5-year-old male; and the viewer D is a 14-year-old female. In other words, in the vicinity of the time t2, the viewers A and B, who are adults, are not suffering from 3D sickness, whereas the viewers C and D, who are minors, are suffering from 3D sickness. In the vicinity of the time t4, only the viewer C is suffering from 3D sickness. In this case, the 3D reproducing unit 13 can, for example, place a priority on the biological signal values associated with the viewers C and D, who are minors, and take a 3D sickness relieving action accordingly.

At time t5, the rates of change in the video and audio signals are small. The 3D content therefore presumably does not particularly show an exciting scene at the time t5. The viewers A to D, however, show large biological signal values. In this case, the 3D reproducing unit 13 takes a 3D sickness relieving action.

To control the sickness detection threshold value properly, the 3D storage medium 12 preferably stores in advance information on exciting scenes and other similar scenes in relation to the time along which 3D content is reproduced. Information representing the positions where exciting scenes in 3D content are reproduced is hereinafter referred to as a viewing pattern.

### [Example of 3D Sickness Detection Using Viewing Pattern]

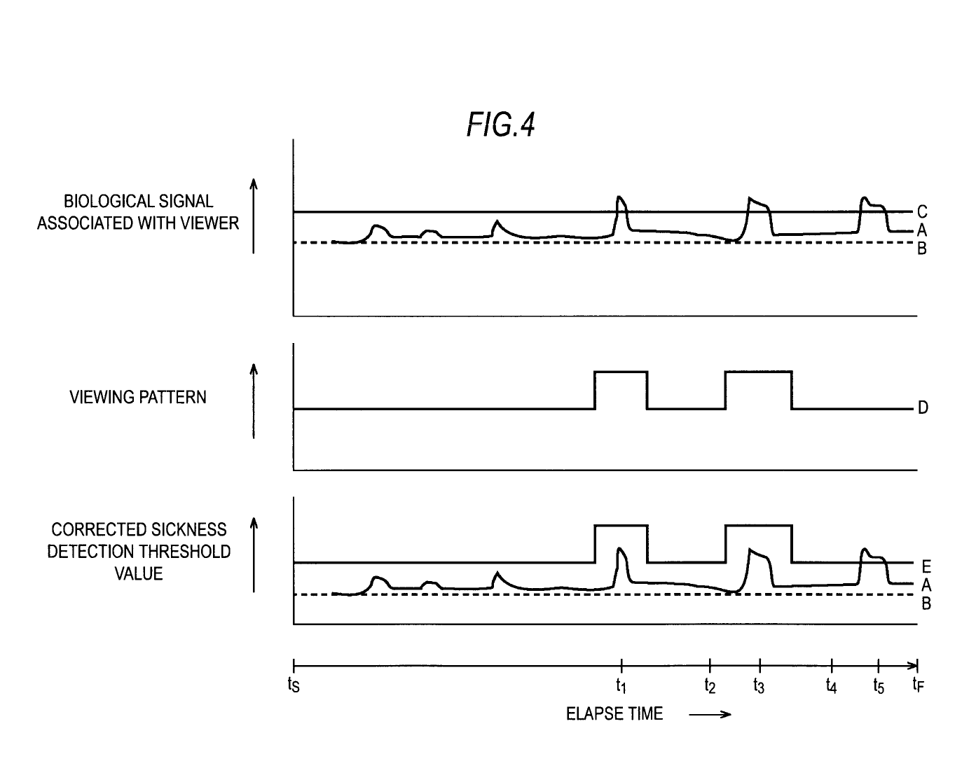

Controlling the sickness detection threshold value using a viewing pattern will next be described with reference to FIG. 4.

The horizontal axis of FIG. 4 represents the time along which 3D content is reproduced, as in FIG. 2. That is, when certain 3D content is reproduced, the reproduction of the 3D content starts from time tS and ends at time tF.

The upper portion of FIG. 4 shows a graph representing a viewer's biological signal versus the time along which the 3D content is reproduced. As an example, the line A represents the biological signal associated with the viewer A described with reference to FIGS. 2 and 3.

The upper portion of FIG. 4 also shows a line B representing a standard value of the biological signal associated with the viewer A. The upper portion of FIG. 4 further shows a line C representing a sickness detection threshold value set to be a value larger than the standard value of the biological signal associated with the viewer A by a predetermined value.

The value of the biological signal (line A) associated with the viewer A is greater than the sickness detection threshold value (line C) in the vicinities of time t1, t3, and t5.

The graph in the middle portion of FIG. 4 shows a viewing pattern of the 3D content versus the time along which the 3D content is reproduced. In the graph in the middle portion of FIG. 4, the ranges of the line D that have larger vertical coordinates represents exciting scenes. That is, the line D shows sections where at least one of the rate of change in brightness (or luminance) of the video signal and the rate of change in the audio signal is larger than a predetermined value.

The lower portion of FIG. 4 shows a graph representing the sickness detection threshold value corrected based on the viewing pattern versus the time along which the 3D content is reproduced.

The line E of the graph in the lower portion of FIG. 4 represents sickness detection threshold values obtained by correcting the sickness detection threshold values (line C) based on the viewing pattern (line D). A sickness detection threshold value thus corrected based on a viewing pattern is hereinafter referred to as a corrected sickness detection threshold value.

Specifically, in the graph in the upper portion of FIG. 4, for example, in the vicinities of the time t1 and t3, the biological signal (line A) associated with the viewer A is greater than the sickness detection threshold value (line C). Compared with the corrected sickness detection threshold value (line E), however, the biological signal (line A) associated with the viewer A is lower than or equal to the corrected sickness detection threshold value (line E). The biological signal processing section 22 therefore judges that the viewer A is not suffering from 3D sickness in the vicinities of the time t1 and t3.

In the vicinity of the time t5, the biological signal (line A) associated with the viewer A is greater than the corrected sickness detection threshold value (line E). The biological signal processing section 22 therefore judges that the viewer A is suffering from 3D sickness in the vicinity of the time t5.

In this way, using corrected sickness detection threshold values corrected based on a viewing pattern allows the present embodiment to provide viewers with a 3D sickness preventive measure adapted to 3D content.

### [Examples of Judgment of 3D Sickness Relieving Action]

When the biological signal processing section 22 detects a viewer who is suffering from 3D sickness, the judgment whether or not a 3D sickness relieving action is taken can be made, for example, as follows:

As a first example, the 3D reproducing unit 13 places a priority on the state of 3D sickness of a viewer who is of a predetermined age or under (minor, for example) among a plurality of viewers and takes a 3D sickness relieving action accordingly, as described above. In this way, a harmful influence of viewing 3D stereoscopic video images on children, whose optic nerves and brain have not been fully developed, can be reduced.

As a second example, the 3D reproducing unit 13 takes a 3D sickness relieving action when at least one of a plurality of viewers is suffering from 3D sickness.

As a third example, the 3D reproducing unit 13 takes a 3D sickness relieving action when a predetermined proportion (50%, for example) of a plurality of viewers is suffering from 3D sickness. The predetermined proportion can be arbitrarily set by the viewers.

As a fourth example, the 3D reproducing unit 13 takes a 3D sickness relieving action when a specific one of a plurality of viewers is suffering from 3D sickness. In this case, viewer information on a specific viewer is stored in advance in the storage section 21. It is thus possible to provide 3D content, for example, in consideration of a viewer having chronic illness and a viewer who tends to suffer from 3D sickness.

When viewers' viewing conditions can be controlled individually, a 3D sickness relieving action is, of course, taken only for an identified viewer who is suffering from 3D sickness.

### [Examples of how to Set Sickness Detection Threshold Value]

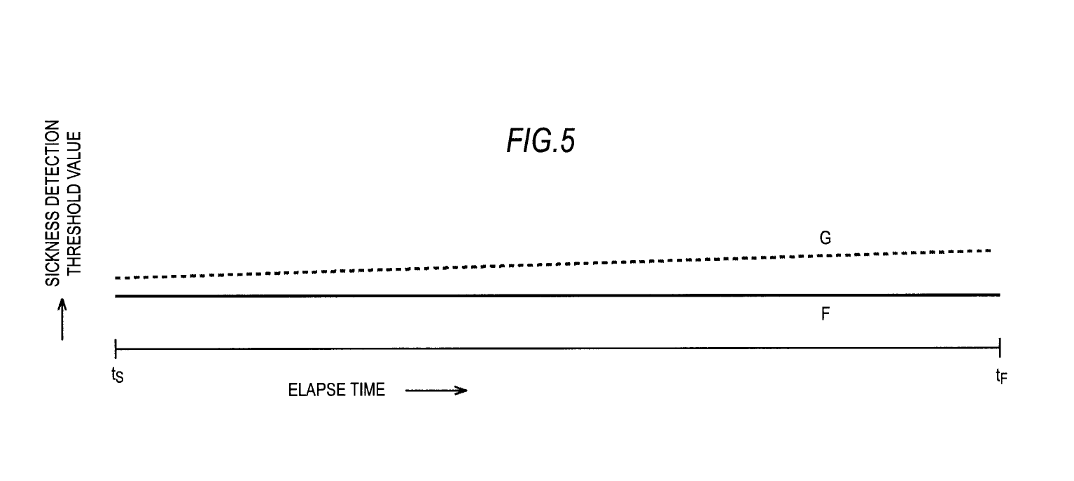

Examples of how to set the sickness detection threshold value will next be described with reference to FIG. 5.

FIG. 5 shows the sickness detection threshold value versus the time along which 3D content is reproduced.

In FIG. 5, the horizontal axis represents the time along which 3D content is reproduced. That is, when certain 3D content is reproduced, the reproduction of the 3D content starts from time tS and ends at time tF. The vertical axis represents the sickness detection threshold value.

The sickness detection threshold value may, for example, be a fixed value over the period from the time when the reproduction of the 3D content starts to the time when the reproduction of the 3D content ends, as indicated by the line F in FIG. 5.

However, for example, a viewer, a small child in particular, may be tired when the viewing period is long. In this case, the sickness detection threshold value can be controlled to increase gradually over the period from the time when the reproduction of the 3D content starts to the time when the reproduction of the 3D content ends, for example, as indicated by the line G in FIG. 5.

Further, the 3D storage medium 12 that stores 3D content may contain information on viewing age limit (parental limit) in some cases. In this case, the viewing age limit can be used to control the sickness detection threshold value in the present embodiment.

### [Description of Processes Performed by 3D Stereoscopic Video Image Apparatus]

A description will next be made of a process of monitoring the state of 3D sickness of viewers and taking a 3D sickness relieving action in accordance with the state of the 3D sickness in the 3D stereoscopic video image apparatus 1 shown in FIG. 1. The process is hereinafter referred to as a 3D sickness monitoring process.

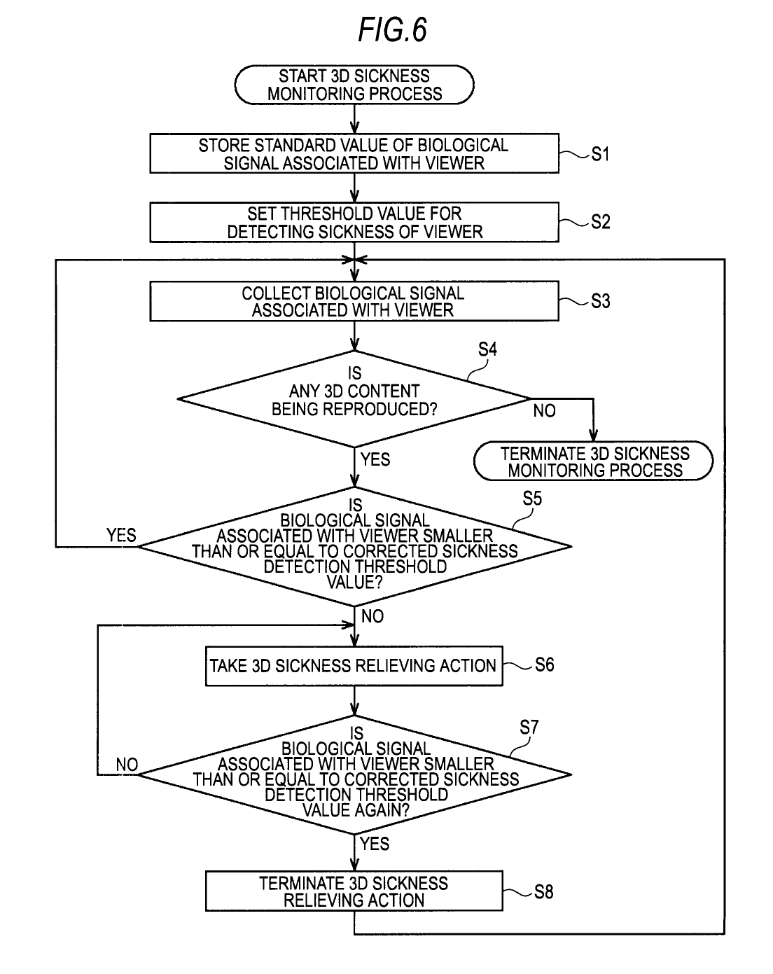

FIG. 6 is a flowchart describing an example of the 3D sickness monitoring process.

As pre-processing before the 3D sickness monitoring process, the storage section 21 stores viewer information on each of the viewers who wear the 3D eyeglasses 16 and the biological signal detectors 17.

In step S1, the 3D reproducing unit 13 stores standard values of biological signals associated with the viewers in the storage section 21. That is, the biological signal processing section 22 acquires biological signals associated with the viewers in a non-3D video image portion from the biological signal detectors 17 via the transceiver 15, and the 3D reproducing unit 13 stores the biological signals as the standard values in the storage section 21. The non-3D video image portion can, for example, be a startup screen on the 3D display 14.

In step S2, the sickness detection threshold value setting section 23 sets a threshold value for detecting sickness of any of the viewers. Specifically, a value larger than the standard values of the viewers' biological signals by a predetermined value is used as the sickness detection threshold value.

In step S3, the 3D reproducing unit 13 collects biological signals associated with the viewers. That is, the biological signal detectors 17 detect the biological signals at all times through polling and send the results to the biological signal processing section 22 via the transceiver 15. In this way, the biological signal processing section 22 collects the viewers' biological signals.

In step S4, the 3D reproducing unit 13 judges whether or not any 3D content is being reproduced.

When no 3D content is being reproduced, the judgment in step S4 shows NO and the 3D sickness monitoring process is terminated.

On the other hand, when certain 3D content is being reproduced, the judgment in step S4 shows YES and the control proceeds to step S5.

That is, the loop from step S3 to step S8 is repeated until the 3D reproducing unit 13 judges that no 3D content is being reproduced, in other words, until the reproduction of the current 3D content ends.

In step S5, the biological signal processing section 22 judges whether or not the viewers' biological signals are smaller than or equal to a corrected sickness detection threshold value. When the biological signal processing section 22 judges that the viewers' biological signals are smaller than or equal to the corrected sickness detection threshold value, the judgment in step S5 shows YES and the control returns to step S3. That is, the loop from step S3 to step S5 is repeated until the biological signal processing section 22 judges that any of the viewers is suffering from 3D sickness.

On the other hand, when the biological signal processing section 22 judges that any of the viewers' biological signals is not smaller than or equal to the corrected sickness detection threshold value, the judgment in step S5 shows NO and the control proceeds to step S6.

In step S6, the 3D reproducing unit 13 takes a 3D sickness relieving action. Specifically, the 3D reproducing unit 13 takes an action of, for example, attenuating the 3D effect displayed on the 3D display 14, switching the display on the 3D display 14 to 2D display, or temporarily halting the display of 3D video images being reproduced.

In step S7, the biological signal processing section 22 judges whether or not the viewers' biological signals are smaller than or equal to the corrected sickness detection threshold value again. When any of the viewers' biological signals is not still smaller than or equal to the corrected sickness detection threshold value, the judgment in step S7 shows NO and the control returns to step S6. That is, the loop from step S6 to step S7 is repeated until the biological signal processing section 22 judges that the viewers' biological signals are smaller than or equal to the corrected sickness detection threshold value again.

On the other hand, when the viewers' biological signals are smaller than or equal to the corrected sickness detection threshold value again, the judgment in step S7 shows YES and the control proceeds to step S8.

In step S8, the 3D reproducing unit 13 terminates the 3D sickness relieving action, and the control returns to step S3.

In this way, the control returns to the step of collecting the viewers' biological signals at all times, and the 3D sickness monitoring process continues until the judgment in step S4 shows that no 3D content is being reproduced.

The 3D sickness monitoring process in the 3D stereoscopic video image apparatus 1 shown in FIG. 1 has been described with reference to FIG. 6.

The present embodiment has been described with reference to the case where the method using a pair of eyeglasses with liquid crystal shutters is used as the method for conveying 3D stereoscopic video images. The invention is, however, not limited to the method for conveying 3D stereoscopic video images using a pair of eyeglasses with liquid crystal shutters. That is, the invention is applicable, of course, to a method using a pair of polarized eyeglasses or any other suitable special eyeglasses and to a method using an autostereoscopic display as long as the system detects viewers' biological signals.

Further, the present embodiment has been described by assuming that the 3D storage medium 12 stores 3D content. The invention is also applicable to a case where 3D content is distributed through broadcasting or over a network.

According to the information processing apparatus described above to which the embodiment of the invention is applied, the following advantageous effects can be provided:

According to the information processing apparatus to which the embodiment of the invention is applied, when a viewer is suffering from 3D sickness, a 3D sickness relieving action can be quickly taken by detecting biological signals associated with the viewers. In this way, a 3D sickness relieving action can be taken even when a viewer himself/herself is not aware of 3D sickness. Further, according to the information processing apparatus to which the embodiment of the invention is applied, the 3D sickness relieving action can be quickly terminated when the viewer recovers from the 3D sickness to a normal state.

According to the information processing apparatus to which the embodiment of the invention is applied, a content producer can collect information obtained by relating a 3D content viewing pattern, viewer information, and viewers' biological signals to one another via the network 11. In this way, the 3D content producer can know the states of the viewers in each scene in the 3D content and make use of them in future 3D content production.

According to the information processing apparatus to which the embodiment of the invention is applied, when a plurality of viewers is viewing 3D content simultaneously, it is possible to place a priority on, for example, the state of 3D sickness of a child and take a 3D sickness relieving action accordingly. In this way, a harmful influence of viewing 3D stereoscopic video images on the development of the brain and optic nerves of the child can be reduced.

According to the information processing apparatus to which the embodiment of the invention is applied, the sickness detection threshold value can be changed over the period during which 3D content is reproduced. In this way, a comfortable 3D content viewing environment can be provided to a viewer who tends to be tired when viewing 3D content for a long time.

The system used herein represents the entire apparatus formed of a plurality of devices and processors.

The invention is not limited to an information processing apparatus capable of reproducing content on a BD but is applicable to a variety of information processing apparatus capable of reproducing content on an optical disc, a magneto-optical disc, a tape medium, and a flash memory medium, and other storage media.

The series of processes described above can be carried out by either hardware or software. To carry out the series of processes by software, a program configuring the software is installed in a computer. The computer may be a computer incorporated into dedicated hardware, a general-purpose personal computer capable of performing a variety of functions by installing a variety of programs, or any other suitable computer.

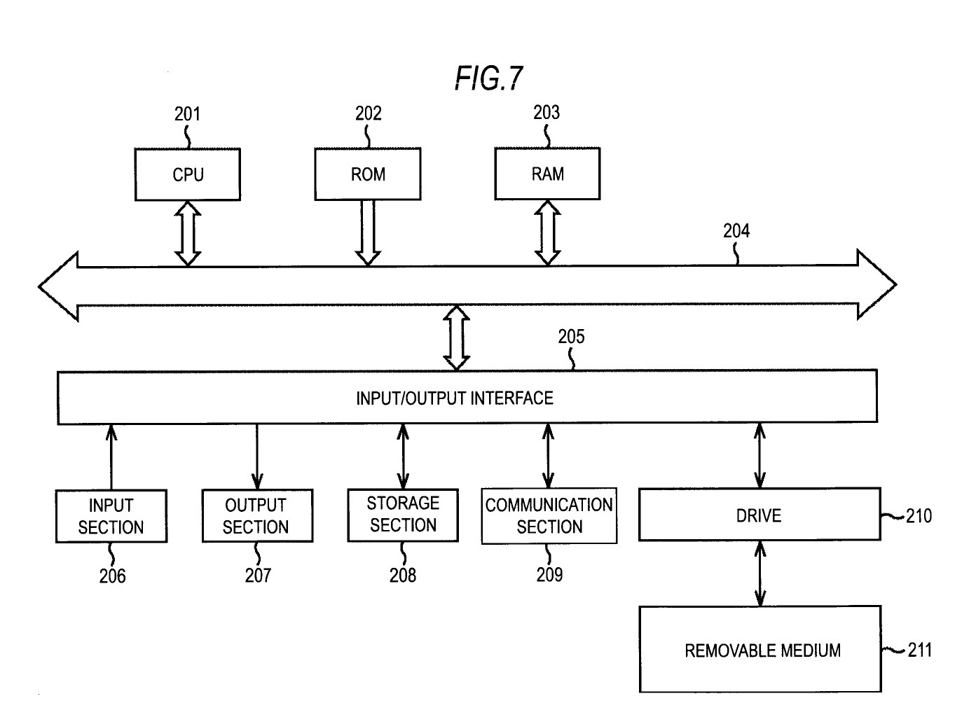

FIG. 7 is a block diagram showing an exemplary configuration of the hardware of a computer that uses a program to carry out the series of processes described above.

In the computer, a CPU 201, a ROM (Read Only Memory) 202, a RAM (Random Access Memory) 203 are interconnected via a bus 204.

An input/output interface 205 is also connected to the bus 204. An input section 206, an output section 207, a storage section 208, a communication section 209, and a drive 210 and a removable medium 211 are connected to the input/output interface 205.

The input section 206 is formed, for example, of a keyboard, a mouse, and a microphone. The output section 207 is formed, for example, of a display and a loudspeaker. The storage section 208 is formed, for example, of a hard disk drive and a non-volatile memory. The communication section 209 is formed, for example, of a network interface. The drive 210 drives the removable medium 211, such as a magnetic disk, an optical disc, a magneto-optical disc, and a semiconductor memory.

In the thus configured computer, the CPU 201, for example, loads a program stored in the storage section 208 into the RAM 203 via the input/output interface 205 and the bus 204 and executes the program to carry out the series of processes described above.

The program to be executed by the computer (CPU 201) can, for example, be recorded on the removable medium 211 and provided as a package medium or the like. The program can also be provided via a wired or wireless transmission medium, such as a local area network, the Internet, and digital satellite broadcasting.

In the computer, the program can be installed in the storage section 208 via the input/output interface 205 by loading the removable medium 211 into the drive 210. The program can alternatively be installed in the storage section 208 by receiving it through the communication section 209 via a wired or wireless transmission medium. Still alternatively, the program can be installed in advance in the ROM 202 or the storage section 208.

The program to be executed by the computer may be a program by which processes are carried out successively in the order described herein or a program by which processes are carried out concurrently or at necessary timings, for example, when the program is called.

The present application contains subject matter related to that disclosed in Japanese Priority Patent Application JP 2009-175442 filed in the Japan Patent Office on Jul. 28, 2009, the entire contents of which is hereby incorporated by reference.

It should be understood by those skilled in the art that various modifications, combinations, sub-combinations and alterations may occur depending on design requirements and other factors insofar as they are within the scope of the appended claims or the equivalents thereof.

What is claimed is:

1. An information processing apparatus comprising:
reproduction means for reproducing 3D content; and
detection means for detecting a biological signal associated with a viewer who is viewing the 3D content,
wherein the reproduction means attenuates the 3D effect of the 3D content when the viewer's biological signal produced when the 3D content is being reproduced exceeds a threshold value.

2. The information processing apparatus according to claim 1,
further comprising storage means for storing a standard value of the viewer's biological signal and viewer information representing the characteristics of the viewer, the standard value and the viewer information related to each other,
wherein the reproduction means changes the threshold value based on a viewing pattern related to a pattern according to which at least one of video and audio signals representing the 3D content changes, the viewing pattern stored in advance on a 3D content storage medium on which the 3D content is stored.

3. The information processing apparatus according to claim 2,
wherein the viewer information contains at least the age of the viewer, and
when a plurality of viewers are viewing the 3D content and the viewer whose biological signal shows a value greater than the threshold value is of a predetermined age or under, the reproducing means places a priority on the viewer and attenuates the 3D effect accordingly.

4. The information processing apparatus according to claim 3,
wherein the reproduction means further changes the threshold value over the period during which the 3D content is reproduced.

5. The information processing apparatus according to claim 4,
wherein the reproduction means transmits information obtained by relating to one another the viewer information, the viewer's biological signal, and the time along which the 3D content is reproduced via a network.

6. An information processing method used with an information processing apparatus including reproduction means and detection means, the method comprising the steps of:
reproducing 3D content by using the reproduction means;
detecting a biological signal associated with a viewer who is viewing the 3D content by using the detection means; and
attenuating the 3D effect of the 3D content by using the reproducing means when the viewer's biological signal produced when the 3D content is being reproduced exceeds a threshold value.

7. A program that instructs a computer to function as:
reproduction means for reproducing 3D content; and
detection means for detecting a biological signal associated with a viewer who is viewing the 3D content,
wherein the reproduction means attenuates the 3D effect of the 3D content when the viewer's biological signal produced when the 3D content is being reproduced exceeds a threshold value.

8. An information processing apparatus comprising:
a reproduction unit configured to reproduce 3D content; and
a detection unit configured to detect a biological signal associated with a viewer who is viewing the 3D content,
wherein the reproduction unit attenuates the 3D effect of the 3D content when the viewer's biological signal produced when the 3D content is being reproduced exceeds a threshold value.
